(19) United States
(12) Patent Application Publication
(10) Pub. No.: US 2022/0409676 A1
(43) Pub. Date: Dec. 29, 2022

(54) COMPOSITIONS COMPRISING BACTERIAL STRAINS FOR USE IN INCREASNG THE BIOAVAILABILITY OF AMINO ACIDS DERIVED FROM PROTEINS

(71) Applicant: SOFAR S.P.A., Trezzano Rosa (IT)

(72) Inventors: Andrea BIFFI, URGNANO (IT); Walter FIORE, TREZZANO ROSA (IT)

(21) Appl. No.: 17/774,432

(22) PCT Filed: Nov. 5, 2020
(86) PCT No.: PCT/IB2020/060412

§ 371 Date: May 4, 2022

(30) Foreign Application Priority Data
Nov. 5, 2019 (IT) 102019000020422

(51) Int. Cl. A61K 35/744; A23L 33/185; A23L 33/135; A23L 33/17; A61K 35/745; A61K 35/747; A61P 1/00; A61P 3/00

(52) U.S. Cl. CPC A61K 35/744; A23L 33/185; A23L 33/135; A23L 33/17; A61K 35/745; A61K 35/747; A61P 1/00; A61P 3/00

(57) ABSTRACT

Compositions are described and related methods and food products for increasing the blood bioavailability of amino acids derived from proteins, preferably proteins of plant origin, based on a mixture M including at least one bacterial strain, preferably Lactobacillus paracasei DG® CNCM I-1572 and/or Lactobacillus paracasei LPC-S01™ DSM 26760. Furthermore, the mixture M is described, as well as the compositions and related methods and food products based on mixture M and further comprises at least one protein, preferably proteins of plant origin, or a peptide or an amino acid.

The present invention relates to compositions for use in increasing the blood bioavailability of amino acids derived from proteins, preferably proteins of plant origin, wherein said compositions comprise a mixture M comprising, or alternatively, consisting of at least one bacterial strain, preferably Lactobacillus paracasei DG® CNCM I-1572 and/or Lactobacillus paracasei LPC-S01™ DSM 26760 (for example, AminoAlta™ trademark registered by Sofar S.p.a., Italy).

Furthermore, the present invention relates to said compositions for use further comprising at least one protein, preferably proteins of plant origin, or a peptide or an amino acid.

Lastly, the present invention relates to a food product comprising proteins, preferably of plant origin, and said mixture M comprising or, alternatively, consisting of at least one bacterial strain, preferably Lactobacillus paracasei DG® CNCM I-1572 and/or Lactobacillus paracasei LPC-S01™ DSM 26760.

Proteins are an essential part of living organisms and they are formed by the union of simpler molecules called amino acids which bind to each other through peptide bonds. Through several reactions, our organism is capable of autonomously synthesising the proteins it needs starting from the individual amino acids contained in food. Many proteins are part of the category of enzymes, whose function is to catalyse the biochemical reactions vital to the metabolism of organisms. Some have structural and mechanical functions, such as actin and myosin in muscles, collagen in bones and tissues, and as components of cell cytoskeleton. Other proteins are important mediators in the transmission of inter- and intra-cellular signals, in immune response, in cell adhesion mechanisms, in the cell-division cycle.

Since proteins cannot be absorbed as such and transported in circulation, some enzymes present in the lumen of the gastrointestinal tract intervene in their digestion breaking them down into individual amino acids. During the digestive process, most proteins are completely reduced into the individual amino acids. Digestion of these macromolecules begins in the stomach, where the combined action of pepsinogen and hydrochloric acid leads to the formation of oligopeptides (short amino acid chains formed by less than ten units). Such digestion is then completed by intestinal proteases of pancreatic origin, which can be divided into endopeptidases and exopeptidases. Specifically, the aforementioned digestion of proteins is completed at the intestinal level thanks to the action of the exopeptidases present in the microvilli of the small intestine, which lead to the formation of the individual amino acids, dipeptides and tripeptides, which can thus be absorbed at the mucosal level by means of an Na+ or H+ transport mechanism.

The intestinal bacterial flora present at the level of the small intestine, especially lactobacilli, also contributes to further digest the peptides, also acting on those not completely hydrolysed by the protease.

Only a small amount of the peptides ingested, equal to about 5%, reaches the colon, where it is subjected to the action of the resident bacterial flora (lactobacilli and bifidobacteria).

Once absorbed, the individual amino acids are transported to the liver by specific carriers and in this case they can:

be used as such and intervene in the immune response, in the synthesis of hormones and vitamins, in the transmission of nerve impulses, in the production of energy and as catalysts in many metabolic processes; participate in protein synthesis, an process inverse to the digestive process that aims at providing the organism with materials for the growth, maintenance and reconstruction of cell structures; if present in excess it can be used for energy purposes (gluconeogenesis) or converted into deposit fat.

The human body breaks down proteins daily, synthesising others. This process is defined as protein turnover. With normal protein intake, only 4% of the proteins turned over may be lost. This situation can be controlled by protein intake, therefore by a high or low supply of daily proteins. The nitrogen balance compares the amount of nitrogen (from food proteins) introduced into the body with the nitrogen being lost. If a person takes more nitrogen than lost, it is said that it results in a positive nitrogen balance, and it deposits nitrogen in the body. If a person takes the same amount of nitrogen as lost, it is said that it results in a balanced nitrogen balance situation, while if a person loses more nitrogen than taken, this is the case of negative nitrogen balance and loses body proteins.

Protein requirement is defined as the amount of food proteins needed to compensate for and equate the loss of nitrogen on a daily basis, so that a person maintains the nitrogen balance. In other words, protein requirement is the amount of protein our body needs to meet its energy needs and maintain good health. These amounts vary depending on some factors such as: age, sex, health condition, work activity or sports activity. The average protein requirement of a person is inversely proportional to age. For example: about 2 g/kg/day in the neonate, about 1.5 g/kg/day at 5 years, and about 1.2 g/kg/day in adolescent-adult age.

Mild protein deficiency can cause: decrease in metabolic efficiency (for example, ease of bleeding, slow wound healing, etc.), decrease in corpusculated elements in the blood, weight loss (as a result of muscle decrease), decrease in muscle volumes, early fatigue, difficulty in concentrating and difficulty in learning, bad mood, muscle and/or joint and/or bone pain, glycemic changes, increased susceptibility to infections. Less frequently, mild protein deficiency can also cause: anxiety (due to the altered synthesis of neurotransmitters), decrease in athletic performance (decrease in compensation of the training stimulus), changes in sleep (some hypothesise that it may be caused by the change in tryptophan and serotonin synthesis), digestive deficiency (proteins allow the natural synthesis of digestive enzymes).

Furthermore, a protein deficiency can generate more serious symptoms or disorders or diseases, such as muscle depletion (consisting of the auto-digestion of muscle proteins to produce energy), decrease in muscle mass and strength and severe decrease in all protein-based components of the organism such as nails, hair, skin, enzymes, neurotransmitters, hormones, immunoglobulins.

A protein is digestible if a high proportion of its amino acids reach the cells of the organism so that they can synthesize the proteins they need.

However, not all proteins are used equally well by the organism, and the bioavailability of the proteins varies depending on the protein source. As a matter of fact, almost 100% of animal proteins is subject to intestinal absorption, whereas plant proteins have a much lower absorption: 52% for lentils, 70% for chickpeas and 36% for wheat.

Furthermore, unlike animal proteins, most plant proteins, such as pea protein, are incomplete because of their low methionine content, and in particular the pea protein contains lower amounts of branched chain amino acids (BCAAs), which play a crucial role in muscle health. Furthermore, plant proteins contain less leucine, one of the essential amino acids for muscle health. This amino acid is particularly important for the activation of muscle protein synthesis. Lastly, it should be pointed out that proteins of plant origin differ in the absorption kinetics and in the amount of amino acids absorbed.

There is currently observed in the population a high consumption of proteins of plant origin, both for ethical reasons and for health reasons, and/or a low consumption of proteins of animal origin. This increased consumption of proteins of plant origin or, in general, a low consumption of proteins of animal origin often does not allow the subject to meet the needed average daily protein requirement.

Furthermore, living conditions that require high protein requirement, such as for example sports activity or pregnancy or breastfeeding, or particular health conditions may require an increased average daily protein requirement of the subject.

Document US2019/0192590 describes the use of probiotic bacterial strains to increase the absorption or the bioavailability of proteins in subjects in need. However, US2019/0192590 neither describes nor discloses an increase in the blood bioavailability of at least one amino acid derived from a protein, even less from a protein of plant origin.

The need therefore remains high to provide an effective solution to meet the average daily protein requirement, in particular in diets with consumption of proteins mainly of plant origin which have a low degree of absorption. In other words, there arises the need for increasing or enhancing the gastrointestinal absorption of proteins and/or protein derivatives, in particular proteins of plant origin, and/or the blood bioavailability of the amino acids derived from said proteins. This need is felt with reference—particularly but not exclusively—to subjects suffering from diseases, disorders and/or symptoms associated with or derived from a protein deficiency, or to subjects in need of a high average protein requirement as well as subjects such as sportsmen/sportswomen, vegetarians and vegans.

In other words, there is a strong interest in the search for nutritional strategies that allow to increase the concentration of amino acids in the blood after ingesting a source of plant proteins, thus allowing to overcome compositional deficiencies.

Such need is even higher in subjects suffering from diseases, disorders and/or symptoms associated with or derived from a protein deficiency or in subjects in need of a high daily average protein need due to their physical condition or physical activity, such as sports subjects, people subjected to high physical stress, subjects with high physical stress, pregnant or breast-feeding women, subjects of geriatric or paediatric age.

Following extensive research and development activity, the Applicant, addresses and solves the aforementioned needs by providing bacterial strains, compositions comprising bacterial strains or food products comprising bacterial strains capable of increasing the gastrointestinal absorption of proteins, preferably proteins of plant origin, and/or the blood bioavailability of amino acids derived from said proteins, in subjects in need, as reported in the present description and in the claims.

Advantageously, the Applicant found that the supplementation of specific probiotics according to the present invention allows to increase the blood absorption and bioavailability of amino acids from proteins, preferably derived from plant proteins.

Advantageously, the bacterial strains, the mixtures, the compositions and the food products of the invention allow to increase the concentration of amino acids in the blood after ingesting a source of plant proteins, thus allowing to overcome compositional deficiencies.

Advantageously, the bacterial strains, mixtures, compositions and food products of the invention allow a higher blood level of amino acids in a subject considering the same intake of proteins and/or protein derivatives, preferably proteins of plant origin.

Advantageously, the bacterial strains, the mixtures, the compositions and the food products of the invention allow to obtain an increase in the maximum concentration (Cmax) and AUC of amino acids, preferably of methionine, histidine, valine, leucine, isoleucine, tyrosine, total BCAAs and maximum concentrations of total EAAs without changing the time required to reach these concentrations (Tmax).

Advantageously, the administration of the bacterial strains, of the mixtures, of the compositions and of the food products of the invention represents an important nutritional strategy for enhancing postprandial changes in blood amino acids and for overcoming the compositional deficiencies of plant proteins.

Advantageously, the bacterial strains, the mixtures, the compositions and the food products of the invention allow a lower consumption of proteins and/or protein derivatives, preferably proteins of plant origin, by a subject to obtain the same protein intake, with the ensuing cost-effectiveness.

Furthermore, the bacterial strains, the mixtures the compositions and food products of the invention do not have significant adverse effects and they can be administered to all subjects, particularly even to paediatric subjects, the elderly, pregnant and breastfeeding women.

Lastly, the compositions of the invention and the products of the invention are effective, easy to prepare and cost-effective.

These and other objects which will be clearer from the detailed description that follows, are achieved by the bacterial strain, by the mixtures and by the compositions of the present invention thanks to the technical characteristics claimed in the attached claims.

### FIGURES

FIG. 1: detailed diagram of the clinical trial.

Forming an object of the present invention is an isolated bacterial strain, or a derivative thereof, for use in increasing the gastrointestinal absorption and/or bioavailability of at least one amino acid derived from a protein of animal or plant origin, preferably a protein of plant origin, in a subject taking—through oral route
said protein, preferably said protein of plant origin, or said at least one amino acid or a peptide comprising said at least one amino acid, wherein said bacterial strain (in short, bacterial strain for use of the invention or a bacterial strain (I) or (I)) is preferably selected from the group comprising or, alternatively, consisting of:
(I.i) a bacterial strain belonging to the species Lactobacillus paracasei identified as Lactobacillus paracasei DG® (trademark registered by SOFAR S.p.A.) and deposited at the National Collection of Cultures of Microorganisms of the Pasteur Institute in Paris under accession number CNCM I-1572 on 5 May 1995 by SOFAR S.p.A. (in short, DG® or L. paracasei DG® CNCM I-1572 or (I.i)); said strain was initially named Lactobacillus casei DG® sub. casei; it was subsequently reclassified as Lactobacillus paracasei DG® CNCM I-1572: it should be observed that it is still and exclusively the same bacterial strain irrespective of the name Lactobacillus casei DG® or Lactobacillus paracasei DG®; and
(I.ii) a bacterial strain belonging to the species Lactobacillus paracasei identified as Lactobacillus paracasei LPC-S01™ and deposited at Deutsche Sammlung von Mikroorganismen und Zellkulturen GmbH (DSMZ) under accession number DSM 26760, by SOFAR S.p.A. on 11 Jan. 2013 for which the conversion to the Budapest Treaty has been filed on 5 May 2017 (in short, LPCS01™ or L. paracasei LPC-S01™ DSM 26760 or (I.ii));
(I.iii) a bacterial strain belonging to the species Bifidobacterium breve identified as Bifidobacterium breve BbIBS01, or a derivative thereof, wherein said bacterial strain was deposited, according to the Budapest Treaty, at Deutsche Sammlung von Mikroorganismen und Zellkulturen GmbH (DSMZ) under deposit number DSM 33231 on 31 Jul. 2019 by Sofar S.p.A.;
(I.iv) a bacterial strain belonging to the species Bifidobacterium breve identified as Bifidobacterium breve BbIBS02, or a derivative thereof, wherein said bacterial strain was deposited at Deutsche Sammlung von Mikroorganismen und Zellkulturen GmbH (DSMZ) under deposit number DSM 33232 on 31 Jul. 2019 by Sofar S.p.A.;
(I.v) a bacterial strain belonging to the species Bifidobacterium animalis identified as Bifidobacterium animalis subsp. lactis BIIBS01, or a derivative thereof, wherein said bacterial strain was deposited at Deutsche Sammlung von Mikroorganismen und Zellkulturen GmbH (DSMZ) under deposit number DSM 33233 on 31 Jul. 2019 by Sofar S.p.A.;
(I.vi) a bacterial strain belonging to the species Lactobacillus plantarum identified as Lactobacillus plantarum LpIBS01, or a derivative thereof, wherein said bacterial strain was deposited at Deutsche Sammlung von Mikroorganismen und Zellkulturen GmbH (DSMZ) under deposit number DSM 33234 on 31 Jul. 2019 by Sofar S.p.A.; and a mixture thereof.

The term “an isolated bacterial strain” is used to indicate a bacterial strain isolated according to standard techniques known to the man skilled in the art.

Forming an object of the present invention is a composition for use in increasing the blood bioavailability of at least one amino acid derived from a protein of plant or animal origin, preferably from a protein of plant origin, in a subject taking—through oral route—said protein or said at least one amino acid or a peptide of plant origin comprising said at least one amino acid, wherein said composition comprises a mixture M comprising, or alternatively, consisting of at least one bacterial strain, or a derivative thereof, selected from the group comprising, or alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM I-1572,
a bacterial strain belonging to the species Lactobacillus paracasei identified as Lactobacillus paracasei LPC-S01® and deposited at Deutsche Sammlung von Mikroorganismen und Zellkulturen GmbH (DSMZ) under accession number DSM 26760,
a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231,
a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232,
a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233,
a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234, and
a mixture thereof; and,

optionally, said composition comprises at least one acceptable pharmaceutical or food grade additive and/or excipient,

wherein said at least one amino acid is selected from the group A comprising or, alternatively, consisting of: arginine, glutamine, citrulline, serine, asparagine, glycine, threonine, alanine, ornithine, methionine, proline, lysine, aspartic acid, histidine, valine, glutamic acid, tryptophan, leucine, phenylalanine, isoleucine, cysteine, tyrosine and mixtures thereof,

and wherein, said increase in the blood bioavailability of said at least one amino acid (expressed as a statistically significant increase in AUC or Cmax) is comprised, preferably from +2% to +55%.

Forming an object of the invention is a composition (in short, composition of the invention) for use in increasing the gastrointestinal absorption and/or the bioavailability of at least one amino acid derived from a protein of animal or plant origin, preferably a protein of plant origin, in a subject taking—through the oral route—said protein, preferably a protein of plant origin or said at least one amino acid or a peptide comprising said at least one amino acid, wherein said composition comprises a mixture M (in short, mixture M of the invention) comprising or, alternatively, consisting of at least one bacterial strain, or a derivative thereof, selected from the group comprising or, alternatively, consisting of: (I.i) Lactobacillus paracasei DG® CNCM I-1572, (I.ii) Lactobacillus paracasei LPC-S01™ DSM 26760, (I.iii) Bifidobacterium breve BbIBS01 DSM 33231, (I.iv) Bifidobacterium breve BbIBS02 DSM 33232, (I.v) Bifidobacterium animalis subsp. lactis BIIBS01 DSM 33233, (I.vi) Lactobacillus plantarum LpIBS01 DSM 33234 and a mixture thereof;

and, optionally, said composition comprises at least one acceptable pharmaceutical or food grade additive and/or excipient.

Furthermore, forming an object of the present invention is a mixture comprising or, alternatively, consisting of, (I.i) Lactobacillus paracasei DG® CNCM I-1572 (freeze-dried in powder form), (I.ii) Lactobacillus paracasei LPC-S01™ DSM 26760 (freeze-dried in powder form), preferably at a 1:1 by weight ratio and, optionally, maltodextrin for use in increasing the blood bioavailability of at least one amino acid derived from a protein of plant or animal origin, preferably from a protein of plant origin, in a subject taking—through oral route—said protein or at least one amino acid or a peptide of plant origin comprising said at least one amino acid.

Furthermore, forming an object of the present invention is probiotic bacterial strain (I.i) Lactobacillus paracasei DG® CNCM I-1572 (freeze-dried in powder form), for use in increasing the blood bioavailability of at least one amino acid derived from a protein of plant or animal origin, preferably from a protein of plant origin, in a subject taking—through oral route—said protein or said at least one amino acid or a peptide of plant origin comprising said at least one amino acid.

Furthermore, forming an object of the present invention is a probiotic bacterial strain (I.ii) Lactobacillus paracasei LPC-S01™ DSM 26760 (freeze-dried in powder form), for use in increasing the blood bioavailability of at least one amino acid derived from a protein of plant or animal origin, preferably from a protein of plant origin, in a subject taking—through oral route—said protein or said at least one amino acid or a peptide of plant origin comprising said at least one amino acid.

Furthermore, forming an object of the present invention is a non-therapeutic use of the composition according to the present invention in increasing the blood bioavailability of at least one amino acid derived from a protein of plant or animal origin, preferably from a protein of plant origin, in a subject taking—through oral route—said protein or said at least one amino acid or a peptide of plant origin comprising said at least one amino acid

Furthermore, forming an object of the present invention is a method for increasing the blood bioavailability of at least one amino acid derived from a protein of plant or animal origin, preferably from a protein of plant origin, comprising administering the composition according to the present invention to a subject taking—through oral route—said protein or at least one amino acid or a peptide of plant origin comprising said at least one amino acid.

In a preferred embodiment of the composition for use of the invention, said mixture M comprises or, alternatively, consists of (I.i) Lactobacillus paracasei DG® CNCM I-1572, or a derivative thereof, and/or (I.ii) Lactobacillus paracasei LPC-S01™ DSM 26760, or a derivative thereof.

In an embodiment of the composition for use of the invention, besides at least one bacterial strain, or a derivative thereof, selected from (1.1) Lactobacillus paracasei DG® CNCM I-1572, (I.ii) Lactobacillus paracasei LPCS01™ DSM 26760 and a mixture thereof, said mixture M further comprises or, alternatively, consists of at least one bacterial strain, or a derivative thereof selected from the group comprising or, alternatively, consisting of: pip Bifidobacterium breve BbIBS01 DSM 33231, (I.iv) Bifidobacterium breve BbIBS02 DSM 33232, (I.v) Bifidobacterium animalis subsp. lactis BIIBS01 DSM 33233, (I.vi) Lactobacillus plantarum LpIBS01 DSM 33234, and mixtures thereof.

In a preferred embodiment of the composition for use of the invention, besides the bacterial strains, or the derivatives thereof, (I.i) Lactobacillus paracasei DG® CNCM I-1572 and (I.ii) Lactobacillus paracasei LPC-S01™ DSM 26760, said mixture M further comprises or, alternatively, consists of at least one bacterial strain, or a derivative thereof, selected from the group comprising or, alternatively, consisting of: (I.iii) Bifidobacterium breve BbIBS01 DSM 33231, (I.iv) Bifidobacterium breve BbIBS02 DSM 33232, (I.v) Bifidobacterium animalis subsp. lactis BIIBS01 DSM 33233, (I.vi) Lactobacillus plantarum LpIBS01 DSM 33234, and mixtures thereof.

In an embodiment of the present invention, the mixture contained in the composition for use according to the present invention comprises, or alternatively, consists of: (I.i) Lactobacillus paracasei DG® CNCM I-1572 (freeze-dried in powder form), (I.ii) Lactobacillus paracasei LPC-S01™ DSM 26760 (freeze-dried in powder form) at a 1:1 by weight ratio and, optionally, maltodextrin.

In an embodiment of the composition for use of the invention, said at least one amino acid, whose blood absorption and/or bioavailability increases due to the effect of said at least one bacterial strain is selected from the group A of amino acids comprising or, alternatively, consisting of: arginine, glutamine, citrulline, serine, asparagine, glycine, threonine, alanine, ornithine, methionine, proline, lysine, aspartic acid, histidine, valine, glutamic acid, tryptophan, leucine, phenylalanine, isoleucine, cysteine, tyrosine and mixtures thereof.

In an embodiment, said increase in the blood bioavailability of said at least one amino acid (expressed as a statistically significant increase in AUC, defined as reported hereinafter in the text) is comprised from +1% to +85%, preferably comprised from +5% to +55%, even more preferably comprised from +15% to +45%, or from 16% to 43%.

In an embodiment, said increase in the blood bioavailability of said at least one amino acid (expressed as a statistically significant increase in Cmax, defined as reported hereinafter in the text) is comprised from +1% to +85%, preferably comprised from +5% to +55%, even more preferably comprised from +15% to +45%, or from 16% to 43%.

Advantageously, said at least one amino acid is selected from the group A.1 of amino acids comprising or, alternatively, consisting of: methionine, histidine, valine, leucine, isoleucine, tyrosine, arginine, cysteine, glycine, glutamine, proline, a mixture of leucine, isoleucine and valine (BCAAs, branched chain amino acids), and a mixture of essential amino acids (EAAs) for humans, consisting of histidine, isoleucine, leucine, lysine, methionine, phenylalanine, threonine, tryptophan, valine,

Advantageously, said increase in blood bioavailability (expressed as statistically significant increase in AUC, defined as reported hereinafter in the text) relates to at least one amino acid selected from group A.1. and it is comprised from +2% to +75%, preferably comprised from +8% to +55%, even more preferably comprised from +15% to +45%, or from 16% to 43%.

Advantageously, said increase in the blood bioavailability (expressed as a statistically significant increase in Cmax, defined as reported hereinafter in the text) relates to at least one amino acid selected from group A.1 and it is comprised from +2% to +85%, preferably from +8% to +55%, even more preferably comprised from +15% to +45%, or from 16% to 43%.

Alternatively, said at least one amino acid is selected from the group A.1.a of amino acids comprising or, alternatively, consisting of: methionine, histidine, valine, leucine, isoleucine, tyrosine, aspartic acid, asparagine, alanine, phenylalanine, tryptophan, total BCAAs, total EAAs and mixtures thereof.

In an embodiment, said increase in blood bioavailability (expressed as statistically significant increase in AUC, defined as reported hereinafter in the text) relates to at least one amino acid selected from the group

A.1.a and it is comprised from +1% to +85%, preferably from +5% to +45%, even more preferably comprised from +15% to +43%, or from 16% to 41%

In an embodiment, said increase in the blood bioavailability (expressed as a statistically significant increase in Cmax, defined as reported hereinafter in the text) relates to at least one amino acid selected from group A.1.a and it is comprised from +1% to +85%, preferably from +5% to +45%, even more preferably from +2% to +52%, or from +10% to +50%.

Even more advantageously, said at least one amino acid is selected from the group A.2 of amino acids comprising or, alternatively, consisting of: methionine, histidine, valine, leucine, isoleucine, tyrosine, aspartic acid, total BCAAs, total EAAs and mixtures thereof.

In an embodiment, said increase in blood bioavailability (expressed as statistically significant increase in AUC, defined as reported hereinafter in the text) relates to at least one amino acid selected from group A.2 and it is comprised from +1% to +85%, preferably comprised from +12% to +45%, even more preferably comprised from +15% to +43%, or comprised from +16% to +43%.

In an embodiment, said increase in the blood bioavailability (expressed as a statistically significant increase in Cmax, defined as reported hereinafter in the text) relates to at least one amino acid selected from group A.2 and it is comprised from +1% to +85%, preferably comprised from +10% to +53%, even more preferably comprised from +11% to +50%, or comprised from +16% to +42%. In the context of the present invention, the term “increasing gastrointestinal absorption and/or bioavailability of amino acids” is used to indicate the increase blood levels (or serum levels) of one or more amino acids in the subject to whom bacterial strains for use of the invention or the composition of the invention are administered.

In the present context, the AUC, Cmax and Tmax parameters are used to assess pharmacokinetic behaviour, specifically the pharmacokinetic behaviour of amino acids in blood.

In the context of the present invention, AUC is used to indicate the area under the concentration/time curve measured using methods known to the man skilled in the art. This parameter is a commonly used parameter for determining blood bioavailability.

AUC is measured (area under the concentration vs. time curve) for each of the 22 amino acids, as well as for BCAAs, EAAs and total amino acids, using the linear trapezoidal rule and using all available time points. Cmax was defined as the maximum observed concentration and Tmax is the time at which Cmax was reached. AUC values were compared under interpolation conditions by means of paired sample t-tests. A p value <0.05 was considered statistically significant. Results are expressed as mean±standard deviation (SD) unless otherwise specified. In vitro digestion data are the results of three independent determinations, each performed in triplicate. Analysis of data variance was performed using the least significant differences (LSD) test.

In the present context, Cmax is used to indicate the maximum observed concentration at a certain time t. This is measured using methods known to the man skilled in the art.

In the present context, Tmax is used to indicate the time at which a maximum concentration (Cmax) is observed. Tmax is measured using methods known to the man skilled in the art.

Advantageously, the bacterial strains, the mixtures, the compositions and the food products of the invention allow to obtain an increase in the Maximum concentration (Cmax) and AUC of the amino acids, without changing the time required to reach such concentrations (Tmax).

Blood bioavailability is expressed as a statistically significant percentage increase in Cmax and/or AUC, and it is defined as the percentage difference measured for each amino acid. Thus, the statistically significant percentage increase intervals of Cmax and/or AUC reported in the present context are to be understood as referring to the single amino acid even if several amino acids are contained in the mixture. For example, in the event of a mixture comprising methionine and histidine the latter may have AUC and Cmax values each comprised within the statistically significant percent increase ranges of Cmax and/or AUC, for example they may be methionine (AUC: +20%, p=0.007; Cmax: +16.3%, p=0.008), histidine (AUC: +40.4%, p=0.009; Cmax: +49.2%, p=0.048).

The blood levels of amino acids are evaluated using analytical methods known to the man skilled in the art. For example, the AUC (area under the curve, as concentration versus time) can be calculated using the linear trapezoidal rule.

Said at least one bacterial strain (I) or a mixture thereof, preferably (I.i) and/or (I.ii), both in the form of strain as such and in the form of composition of the invention, enhances the gastrointestinal absorption and/or increase in bioavailability in the amino acid serum levels when administered associated/combined with proteins, preferably of plant origin, or peptides or amino acids, wherein said administration of said one or more bacterial strains (in short, (I)) and of said proteins, preferably of plant origin, or peptides or amino acids (in short, (II)) is simultaneous or delayed over time, preferably simultaneous over time.

In the context of the present invention, the expression “simultaneous administration over time” of (I) and (II) is used to indicate that (I) and (II) are administered to said subject approximately at the same period of time, in a single composition or product or by means of two distinct compositions or products, or at a distance of a period of time comprised in the range from 5 minutes to 60 minutes, preferably from 5 minutes to 30 minutes, in any order, preferably first taking (I) and then (II).

In the context of the present invention, the expression administration “delayed over time” of (I) and (II) is used to indicate that (I) and (II) are administered to said subject after a period of time from each other by means of two distinct compositions or products, for example at a distance of a period of time comprised in the range from 1 hour to 12 hours, preferably from 1 hour to 6 hours, preferably from 1 hour to 3 hours.

In an embodiment of the composition of the invention, besides the mixture M of the invention comprising or, alternatively, consisting of at least one at least one bacterial strain (I) selected from the group comprising or, alternatively, consisting of (I.i), (I.ii), (I.iv), (I.v), (I.vi) and mixtures thereof or derivatives thereof, preferably (I.i) and/or (I.ii), said composition further comprises at least one protein of animal or plant origin, preferably of plant origin, and/or at least one peptide, preferably of plant origin and/or at least one amino acid; preferably said at least one amino acid comprised in the composition of the invention is selected from group A or group A.1 or A.1.a or group A.2 of amino acids described in the present invention.

In an embodiment of said composition of the invention, said proteins of plant origin are for example proteins obtained from pea, soy bean or lupine, preferably pea, for example yellow pea (Pisum sativum).

Preferably, said at least one bacterial strain (I) or a mixture thereof, preferably (I. 1) and/or (I.ii), for use according to the present invention, is administered to said subject, both in the form of strain as such and in the form of composition of the invention, at a concentration comprised in the range from 10×10⁶ CFU to 10×10¹² CFU, preferably from 10×10⁸ CFU to 10×10¹⁰ CFU, more preferably at a concentration of about 10×10⁹ CFU, with respect to the daily intake (CFU: Colony forming Unit).

In a preferred embodiment of the invention, said bacterial strains (I) of the present invention, preferably (I.i) and/or (I.ii), both as such and comprised in the composition of the invention, are viable bacterial strains, such as, for example, viable bacterial strains present in probiotic products or in Live Biotherapeutic Products (in short, LBP, such as pharmaceutical products comprising viable bacterial strains). “Probiotics” are live and viable micro-organisms (i.e. bacterial strains) which, when administered in adequate amount, confer benefits to the health of the host; the term “probiotics” refers to micro-organisms present in or added to food (FAO and WHO definition).

In a further embodiment of the invention, said bacterial strains (I) of the present invention, preferably (I.i) and/or (I.ii), both as such and comprised in the composition of the invention, is a derivative of a viable bacterial strain.

In the context of the present invention, the term “derivative” of a bacterial strain (or “derivative” of a viable bacterial strain) is used to indicate the bacterial strain tyndallized, or sonicated or inactivated using other techniques known to the man skilled in the art, or lysates of the bacterial strain or extracts of the bacterial strain (paraprobiotics) or any other derivative and/or component of the bacterial strain, preferably exopolysaccharide, parietal fraction, metabolites or metabolic bioproducts generated by the bacterial strain (postbiotics) and/or any other product derived from the bacterial strain. Preferably, the term “derivative” of the bacterial strains of the present invention is used to indicate the tyndallized or inactivated bacterial strain.

Forming an object of the invention is said composition of the invention according to any one of the embodiments of the invention, for use in the preventive and/or curative treatment of a protein deficiency or of an increased protein requirement and/or of a disease, symptom and/or disorder associated with said protein deficiency or increased protein requirement, following increase in gastrointestinal absorption and/or bioavailability of said at least one amino acid.

In an embodiment, said bacterial strain of the invention (I), preferably (I.i) and/or (I.ii), and said composition of the invention according to any one of the embodiments of the invention, allow the treatment of diseases, symptoms and/or disorders associated with said protein deficiency or said increased protein requirement selected from: decrease in metabolic efficiency, decrease in corpusculated elements in the blood, changes in healing processes, decrease in muscle strength, decrease in muscle mass, muscle depletion, weight loss, decrease in athletic performance, early fatigue, difficulty in concentrating, anxiety, changes in sleep, changes in mood, increased susceptibility to infections, digestive deficiency, changes in blood sugar, increased cholesterol, changes in blood-chemical parameters, malnutrition syndrome, such as for example Kwashiorkor or biafra, osteoporosis, decrease in at least one protein-based component of an organism selected from nails, hair, skin, enzymes, neurotransmitters, hormones and immunoglobulins, and other disorders or diseases observable from the context of the present invention.

In an embodiment, said bacterial strain of the invention (I), preferably (I.i) and/or (I.ii), and said composition of the invention according to any one of the embodiments of the invention, are for use as defined in the present invention:
in subjects with increased protein requirement, or,
in subjects with protein deficiency disorders, or,
in paediatric subjects, preferably paediatric subjects with protein deficiency disorders, or,
in pregnant or breastfeeding subjects, preferably pregnant or breastfeeding subjects with protein deficiency disorders, or,
in senile-age subjects, preferably senile-age subjects with protein deficiency, or,
in vegetarian or vegan subjects, preferably vegetarian or vegan subjects with protein deficiency disorders, or,
in subjects receiving therapies which may lead to decrease in protein absorption, or,
in subjects with acute or chronic lesions, for example decubitus ulcers, or,
in sports subjects.

The composition of the invention, comprising said mixture M according to any one of the embodiments of the present invention, further optionally it comprises said at least one pharmaceutical or food grade additive and/or excipient, i.e. a substance devoid of therapeutic activity suitable for pharmaceutical or food use. In the context of the present invention the additives and/or excipients acceptable for pharmaceutical or food use comprise all ancillary substances known to the man skilled in the art for the preparation of compositions in solid, semi-solid or liquid form, such as for example diluents, solvents (including water, glycerine, ethyl alcohol), solubilisers, acidifiers, thickeners, sweeteners, flavour enhancers, colouring agents, lubricants, surfactants, preservatives, stabilisers, pH stabilising buffers and mixtures thereof.

In an embodiment, besides said mixture M of the invention according to any one of the embodiments of the invention, and, optionally, at least one protein or peptide or amino acid, the composition of the present invention may further comprise at least one further active component selected from the group comprising or, alternatively, consisting of other viable bacterial strains and/or parabiotics and/or postbiotics and/or lysates and/or tyndallized and/or inactivated, enzymes, direct or indirect antacid action substances, prebiotic substances, prebiotic substances belonging to the families of yeasts and bacteria, immunostimulatory substances, antidiarrhoea substances, nutrients, vitamins of group B, C, E, organic and/or inorganic salts of magnesium, of selenium, of zinc, antioxidants, anti-radical agents, minerals, plant extracts (botanicals), soy isoflavones.

Said bacterial strains for use of the invention and/or said compositions for use of the invention, may be formulated in solid form, such as tablet, chewable tablet, capsule, lozenge, granules, flakes or powder, in semi-solid form, such as soft-gel, cream, or in liquid form, such as solution, suspension, dispersion, emulsion or syrup.

Said bacterial strains for use of the invention and/or said compositions of the invention, may be formulated for oral (or gastroenteric), sublingual (or buccal), transmucosal, inhalation use (or administration); advantageously they are formulated for oral use, preferably in solid form.

The composition of the invention may be a pharmaceutical composition (or Live Biotherapeutic Products), a medical device composition, a dietary supplement, a food or novel food or probiotic product, a composition for a food for special medical purposes.

In the context of the present invention, the expression “medical device” is used in the meaning according to the Italian Legislative Decree no 46 dated 24 Feb. 1997 or according to the new Medical Device Regulation (EU) 2017/745 (MDR).

Said bacterial strains of the invention or said compositions of the invention may also be for use as adjuvants of further therapeutic approaches, preferably of pharmacological or food type, aimed at treating a protein deficiency and related diseases, symptoms and/or disorders.

Forming an object of the present invention is a method for the preventive or curative treatment, for increasing the gastrointestinal absorption and/or the bioavailability of at least one amino acid derived from a protein of animal or plant origin, preferably a protein of plant origin, in a subject taking—through oral route—said protein, preferably said protein of plant origin, or said at least one amino acid or a peptide comprising said at least one amino acid; in particular for treating diseases, symptoms and/or disorders related with a protein deficiency or related with an increased protein requirement, which provides for the administration—to said subject—a minimum effective amount of at least one bacterial strain (I) of the invention, preferably (I.i) and/or (I.ii), or compositions of the invention according to any one of the embodiments of the present invention.

Forming an object of the present invention is the use of at least one bacterial strain (I) of the invention, preferably (I.i) and/or (I.ii), or compositions of the invention according to any one of the embodiments of the present invention, for increasing the gastrointestinal absorption and/or the bioavailability of at least one amino acid derived from a protein, preferably a protein of plant origin, in a subject taking—through oral route—said protein, preferably said protein of plant origin, or said at least one amino acid or a peptide comprising said at least one amino acid; in particular for the preventive or curative treatment of diseases, symptoms and/or disorders related with protein deficiency or related with increased protein requirement, in a subject.

Forming an object of the present invention is a food or edible product (in short, food or edible product of the invention) comprising proteins of animal or plant origin, preferably proteins of plant origin, and the mixture M of the invention according to any one of the embodiments of the invention, comprising or, alternatively, consisting of at least one bacterial strain or a derivative thereof, selected from the group comprising or, alternatively, consisting of (I.i), (I.ii), (I.iii), (I.iv), (I.v), (I.vi) and a mixture thereof; preferably Lactobacillus paracasei DG® CNCM I-1572 and/or Lactobacillus paracasei LPC-S01™ DSM 26760 and, optionally, at least one of (I.ii), (I.iv), (I.v), (I.vi).

In an embodiment of said food or edible product of the invention, said proteins of plant origin are for example proteins obtained from pea, soy bean or lupine, preferably pea, for example yellow pea (Pisum sativum).

Said food or edible product of the invention may be in solid, liquid or semi-solid form. For example, said food product is a hamburger, preferably a hamburger comprising or, alternatively, consisting of plant proteins.

In an embodiment of said food product of the invention, said at least one bacterial strain (I), preferably (I.i) and/or (I.ii), is in a form that allows to maintain an effectiveness of the bacterial strain to increase the absorption and/or bioavailability of at least one amino acid comprised in said proteins of plant origin following the processes of production and/or processing the food product (for example cooking or high temperatures in general), wherein said form is selected from among the group comprising or, alternatively, consisting of: coated viable bacterial strain, tyndallized bacterial strain, sonicated bacterial strain, inactivated bacterial strain, lysate of the bacterial strain, extract of the bacterial strain, a component of the bacterial strain selected from exopolysaccharide, parietal fraction, metabolite and metabolic bioproduct, in other words a coated probiotic or a paraprobiotic or a postbiotic.

In an embodiment of the invention said food product of the invention is for use for increasing the gastrointestinal absorption and/or the bioavailability of at least one amino acid comprised in said protein of plant origin, as described in the present invention.

Unless otherwise specified, the expression composition or mixture or other comprising a component at an amount “comprised in a range from x to y” is used to indicate that said component may be present in the composition or mixture or extract or other at all amounts present in said range, even if not specified, extremes of the range comprised.

In the context of the present invention, the term “subject/s” is used to indicate human or animal subjects, preferably mammals (e.g. pets such as dogs, cats, horses, sheep or cattle). Preferably, the compositions of the invention are for use in treatment methods for human subjects.

As illustrated in detail in the experimental part, administration—to a subject—of at least one bacterial strain (I) according to the invention increases serum levels of fundamental amino acids, such as methionine, histidine, valine, leucine, isoleucine, tyrosine, total BCAAs and total EMs, in a subject to whom proteins of plant origin were administered.

Thus, the administration of a composition according to the invention (for example, AminoAlta™) can be an important nutritional strategy to improve protein utilisation and overcome the compositional deficiencies of plant proteins.

Preferred embodiments of the present invention FRan are reported below.

FRa1. A composition for use in increasing the blood bioavailability of at least one amino acid derived from a protein of plant origin in a subject who takes said protein of plant origin or said at least one amino acid or a peptide of plant origin comprising said at least one amino acid through oral route, wherein said composition comprises a mixture M comprising, or alternatively, consisting of at least one bacterial strain, or a derivative thereof, selected from the group comprising, or alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM I-1572,
a bacterial strain belonging to the species Lactobacillus paracasei identified as Lactobacillus paracasei LPC-S01™ and deposited at Deutsche Sammlung von Mikroorganismen und Zellkulturen GmbH (DSMZ) under accession number DSM 26760,
a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231,
a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232,
a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233,
a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234, and
a mixture thereof; and,

optionally, said composition comprises at least one acceptable pharmaceutical or food grade additive and/or excipient.

FRa 2. The composition for use according to FRa1, wherein said at least one amino acid is selected from the group A comprising or, alternatively, consisting of: arginine, glutamine, citrulline, serine, asparagine, glycine, threonine, alanine, ornithine, methionine, proline, lysine, aspartic acid, histidine, valine, glutamic acid, tryptophan, leucine, phenylalanine, isoleucine, cysteine, tyrosine and mixtures thereof.

FRa 3. The composition for use according to FRa 1 or 2, wherein said mixture M comprises at least one bacterial strain, or a derivative thereof, selected from the group comprising, or alternatively, consisting of:
Lactobacillus paracasei DG® CNCM I-1572,
Lactobacillus paracasei LPC-S01™ DSM 26760, and a mixture thereof.

FRa 4. The composition for use according to FRa3, wherein said mixture M further comprises at least one bacterial strain, or a derivative thereof, selected from the group comprising, or alternatively, consisting of:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
a mixture thereof.

FRa 5. The composition for use according to any one of FRas 1 to 4, wherein said composition further comprises at least one protein of plant origin, and/or at least one peptide of plant origin, and/or at least one amino acid; preferably wherein said amino acid comprised in the composition is selected from the group A according to claim 2.

FRa 6. The composition for use according to any one of FRas 1 to 5, wherein said subject suffers from a disease, symptom and/or disorder related with a protein deficiency or with an increased protein requirement.

FRa 7. The composition for use according to FRa6, wherein said disease, symptom and/or disorder related with said protein deficiency or with said increased protein requirement is selected from: decrease in metabolic efficiency, decrease in corpusculated elements in the blood, changes in healing processes, decrease in muscle strength, decrease in muscle mass, muscle depletion, weight loss, decrease in athletic performance, early fatigue, difficulty in concentrating, anxiety, changes in sleep, changes in mood, increased susceptibility to infections, digestive deficiency, changes in blood sugar, increased cholesterol, changes in blood-chemical parameters, malnutrition syndrome, such as for example Kwashiorkor or biafra, osteoporosis, decrease in at least one protein-based component of an organism selected from nails, hair, skin, enzymes, neurotransmitters, hormones and immunoglobulins.

FRa 8. A food product comprising
proteins of plant origin, and
the mixture M comprising or, alternatively, consisting of at least one bacterial strain selected from the group comprising or, alternatively, consisting of:
   Lactobacillus paracasei DG® CNCM I-1572,
   Lactobacillus paracasei LPC-S01™ DSM 26760,
   Bifidobacterium breve BbIBS01 DSM 33231,
   Bifidobacterium breve BbIBS02 DSM 33232,
   Bifidobacterium animalis subsp. lactis BIIBS01 DSM 33233,
   Lactobacillus plantarum LpIBS01 DSM 33234, and
   a mixture thereof.

FRa 9. The food product according to FRa 8, wherein said mixture M comprises at least one bacterial strain, or a derivative thereof, selected from the group comprising, or alternatively, consisting of:
Lactobacillus paracasei DG® CNCM I-1572,
Lactobacillus paracasei LPC-S01™ DSM 26760, and
a mixture thereof;

and, optionally, wherein said mixture M further comprises at least one bacterial strain, or a derivative thereof, selected from the group comprising, or alternatively, consisting of:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
a mixture thereof.

FRa 10. The food product according to FRa 8 or 9, wherein said at least one bacterial strain is in a form of coated viable bacterial strain or coated probiotic, paraprobriotic or postbiotic; preferably at least one bacterial strain is in a form selected from the group comprising or, alternatively, consisting of: coated viable bacterial strain, tyndallized bacterial strain, sonicated bacterial strain, inactivated bacterial strain, lysate of the bacterial strain, extract of the bacterial strain, a component of the bacterial strain selected from exopolysaccharide, parietal fraction, metabolite and metabolic bioproduct.

FRa11. The food product according to any one of FRas 8 to 10 for use in increasing the blood bioavailability of at least one amino acid derived from said proteins of plant origin.

Preferred embodiments of the present invention FRbn are reported below.

FRb1. A composition for use in increasing the blood bioavailability of at least one amino acid derived from a protein of plant origin in a subject who takes said protein of plant origin or said at least one amino acid or a peptide of plant origin comprising said at least one amino acid through oral route, wherein said composition comprises a mixture M comprising, or alternatively, consisting of at least one bacterial strain, or a derivative thereof, selected from the group comprising, or alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM I-1572,
a bacterial strain belonging to the species Lactobacillus paracasei identified as Lactobacillus paracasei LPC-S01 ™ and deposited at Deutsche Sammlung von Mikroorganismen und Zellkulturen GmbH (DSMZ) under accession number DSM 26760,
a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231,
a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232,
a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233,
a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234, and
a mixture thereof; and,

optionally, said composition comprises at least one acceptable pharmaceutical or food grade additive and/or excipient, wherein said at least one amino acid is selected from the group A comprising or, alternatively, consisting of: arginine, glutamine, citrulline, serine, asparagine, glycine, threonine, alanine, ornithine, methionine, proline, lysine, aspartic acid, histidine, valine, glutamic acid, tryptophan, leucine, phenylalanine, isoleucine, cysteine, tyrosine and mixtures thereof.

FRb2. The composition for use according to FRb 1, wherein said at least one amino acid is selected from the group A.1.a comprising or, alternatively, consisting of: asparagine, alanine, methionine, aspartic acid, histidine, valine, tryptophan, leucine, phenylalanine, isoleucine, tyrosine and mixtures thereof.

FRb3. The composition for use according to FRb 1 or 2, wherein said mixture M comprises at least one bacterial strain, or a derivative thereof, selected from the group comprising or, alternatively, consisting of:
Lactobacillus paracasei DG® CNCM I-1572,
Lactobacillus paracasei LPC-S01™ DSM 26760, and a mixture thereof, preferably at a 1:1 by weight ratio.

FRb4. The composition for use according to FRb3, wherein said mixture M further comprises at least one bacterial strain, or a derivative thereof, selected from the group comprising or, alternatively, consisting of:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
a mixture thereof.

FRb5. The composition for use according to any one of FRbs 1 to 4, wherein said composition further comprises at least one protein of plant origin, and/or at least one peptide of plant origin, and/or at least one amino acid; preferably wherein said amino acid comprised in the composition is selected from the group A according to claim 2.

FRb6. The composition for use according to any one of FRbs 1 to 5, wherein said subject suffers from disease, symptom and/or disorder associated with a protein deficiency or increased protein requirement.

FRb7. The composition for use according to FRb 6, wherein said disease, symptom and/or disorder associated with said protein deficiency or with said increased protein requirement is selected from: decrease in metabolic efficiency, decrease in corpusculated elements in the blood, changes in healing processes, decrease in muscle strength, decrease in muscle mass, muscle depletion, weight loss, decrease in athletic performance, early fatigue, difficulty in concentrating, anxiety, changes in sleep, changes in mood, increased susceptibility to infections, digestive deficiency, changes in blood sugar, increased cholesterol, changes in blood-chemical parameters, malnutrition syndrome such as Kwashorkor or biafra, osteoporosis, reduction of at least one protein-based component of an organism selected from nails, hair, skin, enzymes, neurotransmitters, hormones and immunoglobulins.

FRb8. A food product comprising
proteins of plant origin, and
the mixture M comprising or, alternatively, consisting of at least one bacterial strain selected from the group comprising or, alternatively, consisting of:
   Lactobacillus paracasei DG® CNCM I-1572,
   Lactobacillus paracasei LPC-S01™ DSM 26760,
   Bifidobacterium breve BbIBS01 DSM 33231,
   Bifidobacterium breve BbIBS02 DSM 33232,
   Bifidobacterium animalis subsp. lactis BIIBS01 DSM 33233,
   Lactobacillus plantarum LpIBS01 DSM 33234, and
   a mixture thereof.

FRb9. The food product according to FRb8, wherein said mixture M comprises at least one bacterial strain, or a derivative thereof, selected from the group comprising or, alternatively, consisting of:
Lactobacillus paracasei DG® CNCM I-1572,
Lactobacillus paracasei LPC-S01™ DSM 26760, and
a mixture thereof;

and, optionally, wherein said mixture M further comprises at least one bacterial strain, or a derivative thereof, selected from the group comprising, or alternatively, consisting of:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
a mixture thereof.

FRb10. The food product according to FRb 8 or 9, wherein said at least one bacterial strain is in a form of a coated viable bacterial strain or coated paraprobiotic or postbiotic, or probiotic; preferably at least one bacterial strain is in a form selected from the group comprising or, alternatively, consisting of: coated viable bacterial strain, tyndallized bacteria strain, sonicated bacterial strain, inactivated bacteria strain, lysate of the bacterial strain, extract of the bacterial strain, a component of the bacterial strain selected from exopolysaccharide, parietal fraction, metabolite and metabolic bioproduct.

FRb11. The food product according to any of FRbs 8 to 10 for use in increasing the blood bioavailability of at least one amino acid derived from said proteins of plant origin.

FRb12. A non-therapeutic use of a composition for increasing the blood bioavailability of at least one amino acid derived from a protein of plant origin in a subject taking—through oral route—said protein of plant origin or said at least one amino acid or a peptide of plant origin comprising said at least one amino acid,

wherein said composition comprises a mixture M comprising, or alternatively, consisting of at least one bacterial strain, or a derivative thereof, selected from the group comprising, or alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM I-1572,
a bacterial strain belonging to the species Lactobacillus paracasei identified as Lactobacillus paracasei LPC-S01™ and deposited at Deutsche Sammlung von Mikroorganismen und Zellkulturen GmbH (DSMZ) under accession number DSM 26760,
a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231,
a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232,
a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233,
a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234, and
a mixture thereof; and,

optionally, said composition comprises at least one acceptable pharmaceutical or food grade additive and/or excipient, wherein said at least one amino acid is selected from the group A comprising or, alternatively, consisting of: arginine, glutamine, citrulline, serine, asparagine, glycine, threonine, alanine, ornithine, methionine, proline, lysine, aspartic acid, histidine, valine, glutamic acid, tryptophan, leucine, phenylalanine, isoleucine, cysteine, tyrosine and mixtures thereof.

FRb13. A method for increasing the blood bioavailability of at least one amino acid derived from a protein of plant origin in a subject taking—through oral route—said protein of plant origin or said at least one amino acid or a peptide of plant origin comprising said at least one amino acid, wherein said method comprises administering a composition comprising a mixture M comprising or, alternatively, consisting of at least one bacterial strain or a derivative thereof, selected from the group comprising or, alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM I-1572,
a bacterial strain belonging to the species Lactobacillus paracasei identified as Lactobacillus paracasei LPC-S01™ and deposited at Deutsche Sammlung von Mikroorganismen und Zellkulturen GmbH (DSMZ) under accession number DSM 26760,
a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231,
a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232,
a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233,
a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234, and
a mixture thereof; and,

optionally, said composition comprises at least one acceptable pharmaceutical or food grade additive and/or excipient, and wherein,

said at least one amino acid is selected from the group A comprising or, alternatively, consisting of: arginine, glutamine, citrulline, serine, asparagine, glycine, threonine, alanine, ornithine, methionine, proline, lysine, aspartic acid, histidine, valine, glutamic acid, tryptophan, leucine, phenylalanine, isoleucine, cysteine, tyrosine and mixtures thereof.

FRb14. A probiotic bacterial strain (I.i) Lactobacillus paracasei DG® CNCM I-1572 (freeze-dried in powder form), for use in increasing the blood bioavailability of at least one amino acid derived from a protein of plant or animal origin, preferably from a protein of plant origin, in a subject taking—through oral route—said protein or at least one amino acid or a peptide of plant origin comprising said at least one amino acid.

FRb15. A probiotic bacterial strain (I.ii) Lactobacillus paracasei LPC-S01™ DSM 26760 (freeze-dried in powder form), for use in increasing the blood bioavailability of at least one amino acid derived from a protein of plant or animal origin, preferably from a protein of plant origin, in a subject taking—through oral route—said protein or at least one amino acid or a peptide of plant origin comprising said at least one amino acid.

### EXPERIMENTAL PART

In Vitro Test

This trial investigated the effect of the individual strains of LP-DG® (CNCM I-1572), and L. paracasei LPC-S01™ (DSM 26760) on the in vitro digestion of amino acids derived from different sources of plant proteins, as well as their synergistic effect when taken in combination.

Suspensions of pea or rice protein isolates were incubated with probiotic bacterial strains (LP-DG® (CNCM I-1572) and/or L. paracasei LPC-S01™ (DSM 26760)) in the presence and in the absence of porcine pepsin (EC 3.4.23.1; Sigma-Aldrich, Milan, Italy) and pancreatin (Sigma-Aldrich, Milan, Italy).

In particular, 0.8 mL of the pea and/or rice protein isolate suspension (10 g/L) was mixed with 0.02 mL of porcine gastric pepsin solution (2 g/L in water). After 1 hour of incubation at 37° C. under mixing, the pH was adjusted to by adding 1 M Tris base and 0.2 mL of porcine pancreas pancreatin (2 g/L in 0.1 M Tris-HCl Buffer, pH 7.5; CE 232-468-9).

Pancreatin digestion was carried out for 3 hours in the absence or in the presence of 0.1 mL of probiotic cells ((LP-DG® (CNCM I-1572), and/or L. paracasei LPC-S01™ (DSM 26760)) (250 g/L in 0.1 M Tris-HCl buffer, pH 7.5).

The peptides released by proteolysis under all the conditions tested were evaluated by adding trichloroacetic acid (TCA) to an aliquot of each reaction mixture (suspension of proteins+pepsin and pancreatin with and without the probiotic strains mentioned above) up to obtaining a final amount of 10% (by volume). After centrifuging at 10000×g for 25 min at room temperature, the absorbance of the supernatant from which the amount of peptides was obtained was measured at 280 nm.

As concerns SDS-PAGE analysis, aliquots of the different reaction mixtures were diluted at various incubation times (1/1, v/v dilution) with Laemmli denaturing buffer (0.125 M Tris-HCl, pH 6.8; 50% glycerol; 1.7% SDS; bromophenol blue at 0.01%; 1% 2-mercaptoethanol) and heated at 100° C. for 10 min. SDS-PAGE was conducted on a 12% monomer gel, using a MiniProtein apparatus (Bio-Rad, Richmond, Va., USA) and the gels were stained with Coomassie Blue (triphenylmethane blue dye). SDS page (sodium dodecyl sulfate polyacrylamide gel electrophoresis) is an analytical technique used to separate proteins based on their molecular weight. When the proteins are separated by electrophoresis through a gel matrix. In SDS-PAGE, the use of sodium dodecyl sulfate (SDS, also known as sodium lauryl sulfate) and polyacrylamide gel largely eliminates the influence of structure and charge and proteins are separated solely based on the length of the polypeptide chain.

Aliquots of the various reaction mixtures were treated with 0.1% trifluoroacetic acid (TFA) and subsequently centrifuged at 12000×g for 20 min. (0.1% trifluoroacetic acid (TFA) (by volume). An aliquot of 0.2 mL of reaction mixture was loaded onto a “Symmetry”C18 (300 Å; 3.5 μm; 2.1 mm×50 mm; Waters, Milan, IT) column, previously equilibrated with 0.1% TFA in distilled water and eluted at a flow rate of 0.8 mL/min, with a linear gradient starting from 5 min after injection up to 60% (v/v) of acetonitrile, in the presence of TFA at 0.1% (by volume). The chromatographic separation was carried out with a Waters™ 626 equipment provided with a Waters™ 2487 dual wavelength detector (Waters, Milan, Italy). Data at 220 nm and 280 nm were recorded and processed using Empower Pro software (Waters, Milan, Italy). The results are expressed as the total area of the peak measured at 220 nm including peptides containing no aromatic residues which were quantified by measuring the absorbance—at 280 nm—of the supernatant after treatment with TFA.

Results

RP-HPLC analysis showed in particular that L. paracasei LP-DG® showed a particular proteolytic activity for rice protein isolates. In addition, the combination of two strains (LP-DG® (CNCM I-1572) and L. paracasei LPC-S01™ (DSM 26760)) showed a synergistic effect, particularly toward pea proteins (Table 5)

Clinical Trial in Humans The purpose of the clinical trial reported below is to examine the effect of ingesting a probiotic combined with a protein (of plant and animal origin) on the subsequent appearance of amino acids in the blood.

Trial Design:

two separate, randomised, double-blind, placebo-controlled crossover designs.

Twenty (n=20) male subjects were randomly assigned to ingest a supplement containing a protein (of plant origin (20 g of pea protein), n=10+5 (see below); or of animal origin, n=10) and a placebo, or a protein (of plant origin (20 g of pea protein), n=10; or of animal origin, n=10) plus a composition according to the invention (5 billion CFU L. paracasei LP-DG® (CNCM I-1572) plus 5 billion CFU L. paracasei LPC-S01™ (DSM 26760), SOFAR S.p.A., Italy)) (in short, probiotic) for two weeks prior to the experimental test.

The daily diet was recorded and the subjects were requested to repeat the diet for the two weeks leading to the second experimental test.

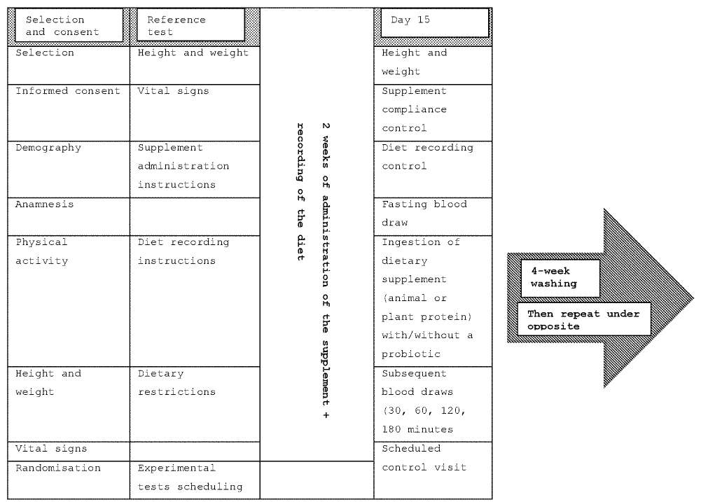

On the day of the experimental tests, subjects were placed under fasting for at least 12 hours. Subjects were catheterised with a teflon catheter in an antecubital vein for multiple blood draws. The catheter is washed with 2-3 ml of 0.9% sodium chloride. Following basic sampling, the subjects were administered with their respective supplement (ingestion of supplement). Subsequently, blood samples were taken at 30, 60, 120 and 180 minutes after ingestion. Subsequently, a four-week wash-out period was implemented, followed by the opposite condition. The expression opposite condition is used to indicate that a subject taking proteins and placebo in the first cycle will take proteins and probiotics in the second round. FIG. 1 provides a detailed diagram of the trial. Blood was collected and transferred into 8.5 ml Becton Dickinson (BD) test tubes (BD Vacutainer SST) to obtain serum, and into 10.0 ml BD tubes (Vacutainer Sodium Heparin) to obtain plasma and subsequently centrifuged at 1500×g for 15 min at 4° C. The resulting serum and plasma were then split into aliquots and stored at −80° C. until subsequent tests.

Subject Population:

Twenty (n=20) normal weight (body mass index (BMI), 19-24.99 kg×m²) male subjects active at recreational level (according to the American College of Sports Medicine guidelines), normal weight (body mass index (BMI), 19-24.99 kg×m²), aged 20 to 35 years.

The twenty subjects were randomly assigned to the plant protein group (n=10) or animal protein group (n=10). Other 5 subjects were added to the group of 10 subjects assigned to the plant protein group for a total of 15 subjects on whom the statistical analysis reported in the present context is based. The narrow age group (20-35 years) was chosen as changes in gut microbiota and potentially digestive enzymes could occur over time with age. This ensures consistency among subjects. The subject will not have taken any known nutritional or ergogenic supplements known to be capable of affecting the current trial measures for the previous 6 weeks, nor will they have any medical condition or allergy capable of affecting the outcome of interest.
Material: proteins of plant origin: protein isolated from yellow pea Nutralys® S85F Roquette Freres S.A., France (Pisum Sativum), in powder form, loss on drying 10% max., protein content on dry base 84% min
Analysis of the amino acid profile of the protein supplement is reported in table 1c below

| Amino acid | mg/g protein |
|---|---|
| alanine | 43 |
| arginine | 88 |
| Aspartic acid | 117 |
| cysteine | 9 |
| Glutamic acid | 167 |
| glycine | 41 |
| histidine | 25 |
| isoleucine | 48 |
| leucine | 83 |
| lysine | 74 |
| methionine | 12 |
| phenylalanine | 54 |
| proline | 45 |
| serine | 50 |
| threonine | 40 |
| tryptophan | 10 |
| tyrosine | 39 |
| valine | 51 |

probiotic (composition according to the invention, AminoAlta™): mixture of (1.1) Lactobacillus paracasei DG® CNCM I-1572 5 billion CFU (freeze-dried in powder form), (I.ii) Lactobacillus paracasei LPC-S01™ DSM 26760 billion CFU (freeze-dried in powder form) and, optionally maltodextrin, according to Table 1a and Table 1b.
Placebo: Maltodextrin (Glucidex® 12, Roquette Freres S.A., France)

The materials to be administered, listed above, were supplied in a sealed sachet separated from each other until the time of consumption. Subjects were instructed to open the sachets and mix the content (protein and placebo, or protein with probiotic) with their favourite non-protein beverage (473 ml).

TABLE 1a

| | | |
|---|---|---|
| Ingredients list | Amt per 100 g | Amt per unit |

| | | | | |
|---|---|---|---|---|
| Maltodextrin | 92.500 | g | 1850.000 | mg |
| Lactobacillus Paracasei | 3.750 | g | 75.000 | mg |
| LPC-S01 ™ DSM 26760 | | | | |
| Lactobacillus Paracasei | 3.750 | g | 75.000 | mg |
| DG ® CNCM I-1572 | | | | |
| Total | 100.000 | g | 2000.000 | mg |

TABLE 1b
NUTRIENT SUPPLY

| Average content | per 100 g | Per daily dose |
|---|---|---|
| Total count of ferments | 1500.000 bln. C.F.U.* | 30.000 bln. C.F.U.* |
| of which Lactobacillus | 750.000 bln. C.F.U.* | 15.000 bln. C.F.U.* |
| Paracasei LPC-S01 ™ | | |
| of which Lactobacillus | 750.000 bln. C.F.U.* | 15.000 bln. C.F.U.* |
| Paracasei DG ® CNCM | | |
| I-1572 | | |

*Amount of ferments present in formula

Amino Acid Analysis

EZfaast® amino acid analysis kits (Phenomenex, Torrance, CA) (US Patent U.S. Pat. No. 6,770,246) were used for liquid chromatographic analysis of amino acids using tandem mass spectrometry (LC/MS/MS) and electrospray ionization (ESI). The procedure consists of solid phase extractions of 25 μl plasma with internal standards with an absorbent tip attached to a syringe with an elution solvent (a 3:2 mixture of sodium hydroxide with 77% n-propanol and 23% 3-picoline). The free amino acids were then derivatized by adding a mixture of 17.4% propyl chloroformate, 11% isooctane and 71.6% chloroform (by volume). The resulting mixture was vortexed and allowed to stand at room temperature for 1 minute, followed by liquid-liquid extraction with isooctane. Fifty microliters of the organic layer were removed, dried under nitrogen flow and suspended in the HPLC run solvents before being injected into the LC/MS/MS. Chromatographic separation of the derivatized amino acids was carried out on an amino acid analysis-mass spectrometry column (250×2.0 mm d.i., 4 μm) using a triple quadrupole Agilent 6460 LC/MS/MS system (Santa Clara, Calif.). Ten millimetres of ammonium formate in water with 0.2% formic acid (by volume) (mobile phase A) and 10 mm of ammonium formate in methanol with 0.2% formic acid (by volume) (mobile phase B) were used as solvent with a gradient from 68% B at 0 min to 83% B over 13 min with a flow rate of 0.25 ml/min. Amino acids and internal standard data were collected using the dynamic multiple reaction monitoring mode using MassHunter acquisition software (Agilent, Santa Clara, Calif.). MassHunter quantification software was used quantify plasma samples based on standard curves.

Result Variables:

amount of amino acids (i.e. 3-methylhistidine, arginine, glutamine, citrulline, serine, asparagine, glycine, threonine, alanine, ornithine, methionine, proline, lysine, aspartic acid, histidine, valine, glutamic acid, tryptophan, leucine, phenylalanine, isoleucine, cysteine and tyrosine) in peripheral blood measured by means of:
maximum concentration (Cmax);
the corresponding time (Tmax) and area under the curve (AUC), calculated as appropriate.

For statistical analysis, the area under the curve (concentration vs. time, in short AUC) was calculated for each of the 22 amino acids, as well as BCAAs, essential amino acids (EAAs) and total amino acids, through the linear trapezoidal rule and using all available time points. Cmax was defined as the highest observed concentration and Tmax was the time when Cmax was reached. AUC values were compared between conditions through t-test for paired samples. A p value <0.05 was considered statistically significant.

Statistical Analysis:

two separate analyses of two-way repeated measures (subjects administered with proteins of plant origin and with proteins of animal origin) of variance are used to determine differences in result variables. Significance was set to an alpha level of 0.05. When a significant major effect and/or interaction was observed, a post-hoc analysis was performed using Bonferronni correction. Furthermore, magnitude-based inference was used.

Contrary to the null-based hypothesis test (p-value), magnitude-based inference allows an analysis of the response based on clinical significance.

Results:

Tables (2), (3) and (4) report the results of the statistical analysis of the subjects (n=10) participating in the clinical trial administered with protein of plant origin, or with protein of plant origin plus probiotic (composition according to the invention).

In particular,
Table 2 reports the total concentration data (AUC, area under the curve) for the amino acids analysed;
Table 3 reports the Cmax (maximum concentration) data for the amino acids analysed;
Table 4 reports the data of Tmax (in minutes) (time at which the maximum concentration is recorded) for the amino acids analysed.

The statistical analysis revealed—in subjects who took plant proteins plus probiotic—both a higher maximum concentration (Cmax) and a higher total concentration (AUC) for methionine, histidine, valine, leucine, isoleucine, tyrosine, total BCAAs and total EAAs.

In particular, the administration of the composition of the invention comprising the aforementioned bacterial strains (AminoAlta™) resulted in a statistically significant increase in AUC and Cmax for methionine (AUC: +20%, p=0.007; Cmax: +16.3%, p=0.008), histidine (AUC:+40.4%, p=0.009; Cmax: +49.2%, p=0.048), valine (AUC:+21.5%, p=0.013; Cmax: +24.7%, p=0.034), leucine (AUC:+23.3%, p=0.006; Cmax: +25.2%, p=0.043), isoleucine (AUC:+26.0%, p=0.017; Cmax: +26.1%, p=0.020), tyrosine (AUC:+16.0%, p=0.009; Cmax: +11.6, p=0.014), total BCAAs (AUC:+22.8%, p=0.008; Cmax: +26.8, p=0.009) and total EAAs (AUC:+16.0%, p=0.005; Cmax: +15.6, p=0.022) without significantly modifying the time to reach maximum concentrations.

Furthermore, no significant changes were observed between the Tmax of subjects administered with plant protein and the Tmax of subjects administered with the plant protein plus probiotic.

### CONCLUSIONS

Thus, the administration of the composition according to the invention (AminoAlta™) can be an important nutritional strategy to improve protein utilisation and overcome the compositional deficiencies of plant proteins.

TABLE 2

| | AUC | | | | | | |
|---|---|---|---|---|---|---|---|
| | Protein + Probiotic | | Protein | | Protein + Probiotic vs Protein | | |
| | Mean value | SD | Mean value | SD | p-value (t-test) | % Difference | Effect Size (d) |
| Arginine | 14490.3 | 4677.7 | 13954.3 | 6438.0 | 0.585 | 3.8 | 0.10 |
| Glutamine | 90876.4 | 21491.3 | 87569.0 | 25645.0 | 0.649 | 3.8 | 0.14 |
| Citrulline | 3936.4 | 1411.6 | 3922.3 | 1906.1 | 0.967 | 0.4 | 0.01 |
| Serine | 17569.1 | 4194.0 | 17464.6 | 6513.0 | 0.942 | 0.6 | 0.02 |
| Asparagine | 13305.5 | 3226.3 | 12212.4 | 3774.1 | 0.337 | 9.0 | 0.31 |
| Glycine | 33083.9 | 9728.7 | 32626.4 | 10309.8 | 0.874 | 1.4 | 0.05 |
| Threonine | 22917.0 | 4289.4 | 21905.7 | 6917.8 | 0.621 | 4.6 | 0.18 |
| Alanine | 56904.7 | 13342.8 | 53750.8 | 20418.2 | 0.496 | 5.9 | 0.18 |
| Ornithine | 10060.4 | 3934.2 | 9793.9 | 3678.8 | 0.693 | 2.7 | 0.08 |
| Methionine | 3558.9 | 1462.0 | 2966.9 | 1245.0 | 0.007 | 20.0 | 0.44 |
| Proline | 43142.9 | 10833.0 | 42124.2 | 19636.2 | 0.780 | 2.4 | 0.06 |
| Lysine | 36177.2 | 5057.1 | 35387.7 | 10758.1 | 0.759 | 2.2 | 0.09 |
| Aspartic ac. | 711.7 | 259.4 | 591.1 | 277.9 | 0.125 | 20.4 | 0.45 |
| Histidine | 14588.0 | 4519.9 | 10248.1 | 3983.4 | 0.009 | 40.4 | 0.97 |
| Valine | 65216.4 | 17782.9 | 53682.1 | 17525.3 | 0.013 | 21.5 | 0.65 |
| Glutamic ac. | 6523.0 | 3703.0 | 6384.9 | 3079.4 | 0.849 | 2.2 | 0.04 |
| Tryptophan | 13056.3 | 4319.6 | 12078.2 | 3535.2 | 0.276 | 8.1 | 0.25 |
| Leucine | 26443.5 | 10874.2 | 21447.6 | 10701.1 | 0.006 | 23.3 | 0.46 |
| Phenylalanine | 8879.6 | 2629.0 | 8061.8 | 3371.5 | 0.086 | 10.1 | 0.27 |
| Isoleucine | 19048.2 | 6642.5 | 15116.5 | 5961.5 | 0.017 | 26.0 | 0.62 |
| Cysteine | 3016.2 | 932.8 | 3024.9 | 1468.0 | 0.978 | −0.3 | −0.01 |
| Tyrosine | 10790.8 | 3532.4 | 9306.0 | 3923.5 | 0.009 | 16.0 | 0.40 |
| Total BCAAs | 110886.8 | 33142.0 | 90304.5 | 33001.5 | 0.008 | 22.8 | 0.62 |
| Total EAAs | 210115.0 | 41010.5 | 181071.7 | 54739.9 | 0.005 | 16.0 | 0.60 |
| Total AAs | 515152.2 | 79378.2 | 474059.9 | 139979.5 | 0.136 | 8.7 | 0.36 |

TABLE 3

| | Cmax | | | | | | |
|---|---|---|---|---|---|---|---|
| | Protein + Probiotic | | Protein | | Protein + Probiotic vs Protein | | |
| | Mean value | SD | Mean value | SD | p-value (t-test) | % Difference | Effect (d) |
| Arginine | 102.4 | 30.9 | 106.6 | 42.9 | 0.556 | −3.9 | −0.11 |
| Glutamine | 601.7 | 131.8 | 599.9 | 131.2 | 0.974 | 0.3 | 0.01 |
| Citrulline | 26.4 | 9.7 | 27.4 | 10.5 | 0.580 | −3.6 | −0.10 |
| Serine | 128.3 | 28.4 | 130.4 | 47.5 | 0.860 | −1.6 | −0.05 |
| Asparagine | 101.3 | 22.6 | 96.1 | 31.0 | 0.602 | 5.5 | 0.19 |
| Glycine | 228.3 | 56.7 | 237.3 | 66.5 | 0.625 | −3.8 | −0.15 |
| Threonine | 161.9 | 30.5 | 159.2 | 51.0 | 0.861 | 1.7 | 0.06 |
| Alanine | 414.6 | 148.2 | 389.6 | 135.3 | 0.418 | 6.4 | 0.18 |
| Ornithine | 72.8 | 23.4 | 72.0 | 23.7 | 0.881 | 1.2 | 0.04 |
| Methionine | 23.2 | 8.9 | 19.9 | 6.3 | 0.008 | 16.8 | 0.42 |
| Proline | 318.7 | 129.1 | 323.0 | 176.2 | 0.905 | −1.3 | −0.03 |
| Lysine | 276.0 | 45.9 | 266.0 | 70.5 | 0.627 | 3.7 | 0.17 |
| Aspartic ac. | 7.3 | 3.6 | 5.9 | 3.6 | 0.208 | 22.7 | 0.37 |
| Histidine | 110.0 | 59.1 | 73.7 | 24.5 | 0.048 | 49.2 | 0.80 |
| Valine | 490.5 | 174.5 | 393.4 | 94.2 | 0.034 | 24.7 | 0.69 |
| Glutamic ac. | 50.4 | 24.3 | 51.9 | 22.5 | 0.740 | −3.0 | −0.07 |
| Tryptophan | 90.8 | 24.7 | 88.7 | 19.7 | 0.796 | 2.4 | 0.10 |
| Leucine | 200.7 | 80.3 | 160.3 | 71.6 | 0.043 | 25.2 | 0.53 |
| Phenylalanine | 57.6 | 16.8 | 54.8 | 18.9 | 0.252 | 5.2 | 0.16 |
| Isoleucine | 149.4 | 46.8 | 118.4 | 41.6 | 0.020 | 26.1 | 0.70 |
| Cysteine | 19.5 | 5.3 | 19.8 | 7.5 | 0.832 | −1.8 | −0.06 |
| Tyrosine | 70.6 | 23.2 | 63.3 | 22.6 | 0.014 | 11.6 | 0.32 |
| Total BCAAs | 833.8 | 237.5 | 657.8 | 192.6 | 0.009 | 26.8 | 0.81 |
| Total EAAs | 1512.6 | 315.7 | 1308.2 | 301.3 | 0.022 | 15.6 | 0.66 |
| Total AAs | 3566.6 | 586.2 | 3389.4 | 818.6 | 0.371 | 5.2 | 0.25 |

TABLE 4

| | Tmax | | | | | | |
|---|---|---|---|---|---|---|---|
| | Protein + Probiotic | | Protein | | Protein + Probiotic vs Protein | | |
| | Mean value | SD | Mean value | SD | p-value (t-test) | % Difference | Effect (d) |
| Arginine | 58.0 | 41.6 | 40.0 | 14.5 | 0.057 | 45.0 | 0.58 |
| Glutamine | 54.0 | 41.2 | 60.0 | 56.7 | 0.748 | −10.0 | −0.12 |
| Citrulline | 56.0 | 46.6 | 48.0 | 49.2 | 0.546 | 16.7 | 0.17 |
| Serine | 52.0 | 40.0 | 40.0 | 29.3 | 0.271 | 30.0 | 0.34 |
| Asparagine | 58.0 | 43.1 | 46.0 | 33.8 | 0.395 | 26.1 | 0.31 |
| Glycine | 70.0 | 55.2 | 52.0 | 56.1 | 0.398 | 34.6 | 0.32 |
| Threonine | 62.0 | 51.3 | 50.0 | 40.4 | 0.458 | 24.0 | 0.26 |
| Alanine | 78.0 | 54.1 | 56.0 | 49.3 | 0.294 | 39.3 | 0.43 |
| Ornithine | 72.0 | 45.1 | 52.0 | 33.0 | 0.096 | 38.5 | 0.51 |
| Methionine | 48.0 | 42.1 | 38.0 | 13.7 | 0.388 | 26.3 | 0.32 |
| Proline | 66.0 | 53.4 | 56.0 | 54.2 | 0.632 | 17.9 | 0.19 |
| Lysine | 50.0 | 40.4 | 54.0 | 44.2 | 0.784 | −7.4 | −0.09 |
| Aspartic ac. | 46.0 | 35.6 | 50.0 | 43.4 | 0.774 | −8.0 | −0.10 |
| Histidine | 62.0 | 51.3 | 44.0 | 43.7 | 0.246 | 40.9 | 0.38 |
| Valine | 60.0 | 40.9 | 58.0 | 53.7 | 0.914 | 3.4 | 0.04 |
| Glutamic ac. | 42.0 | 37.3 | 74.0 | 69.8 | 0.088 | −43.2 | −0.57 |
| Tryptophan | 52.0 | 43.1 | 72.0 | 54.1 | 0.191 | −27.8 | −0.41 |
| Leucine | 60.0 | 40.9 | 44.0 | 15.5 | 0.120 | 36.4 | 0.52 |
| Phenylalanine | 52.0 | 24.0 | 42.0 | 15.2 | 0.096 | 23.8 | 0.50 |
| Isoleucine | 52.0 | 38.4 | 44.0 | 15.5 | 0.413 | 18.2 | 0.27 |
| Cysteine | 54.0 | 45.6 | 72.0 | 58.7 | 0.447 | −25.0 | −0.34 |
| Tyrosine | 58.0 | 36.7 | 46.0 | 15.5 | 0.233 | 26.1 | 0.43 |
| Total BCAAs | 66.0 | 42.7 | 54.0 | 39.5 | 0.458 | 22.2 | 0.29 |
| Total EAAs | 60.0 | 40.9 | 54.0 | 39.5 | 0.689 | 11.1 | 0.15 |
| Total AAs | 58.0 | 43.1 | 60.0 | 52.0 | 0.910 | −3.3 | −0.04 |

TABLE 5
The data are presented as means ± standard deviation

| | Rice | | Pea | |
|---|---|---|---|---|
| Treatment | Area (*10⁶) | Δ, % | Area (*10⁶) | Δ, % |

Absorbance at 220 nm

| | | | | |
|---|---|---|---|---|
| Pepsin + pancreatin | 78.2 ± 0.10 | | 107 ± 0.60 | |
| Pepsin + pancreatin + L. paracasei LP-DG | 80.2 ± 0.15* | +2.6 | 107 ± 0.50 | 0 |
| Pepsin + pancreatin + L. paracasei LPC-S01 | 78.4 ± 0.21 | +0.3 | 111 ± 0.80* | +3.7 |
| Pepsin + pancreatin + LP-DG + LPC-S01 | 80.6 ± 0.18* | +3.1 | 114 ± 0.70** | +6.5 |

Absorbance at 280 nm

| | | | | |
|---|---|---|---|---|
| Pepsin + pancreatin | 5.1 ± 0.10 | | 11.9 ± 0.06 | |
| Pepsin + pancreatin + L. paracasei LP-DG | 5.4 ± 0.09* | +5.9 | 12.0 ± 0.08 | +0.8 |
| Pepsin + pancreatin + L. paracasei LPC-S01 | 5.2 ± 0.08 | +2.0 | 12.2 ± 0.02* | +2.5 |
| Pepsin + pancreatin + LP-DG + LPC-S01 | 5.4 ± 0.10* | +5.9 | 12.6 ± 0.07** | +5.6 |

*significantly different from control (p < 0.05)
**significantly different from control and individual strain (p < 0.05)

1. A composition for increasing blood bioavailability of at least one amino acid derived from a protein of plant origin in a subject taking said protein of plant origin or said at least one amino acid or a peptide of plant origin comprising said at least one amino acid through oral route,
said composition comprising
a mixture M in an effective amount to increase bioavailability of the at least one amino acid, the at least one amino acid selected from arginine, glutamine, citrulline, serine, asparagine, glycine, threonine, alanine, ornithine, methionine, proline, lysine, aspartic acid, histidine, valine, glutamic acid, tryptophan, leucine, phenylalanine, isoleucine, cysteine, tyrosine and mixtures thereof, the mixture M comprising at least one bacterial strain, or a derivative thereof, selected from:
Lactobacillus paracasei DG® deposited at the National Collection of Cultures of Microorganisms of the Pasteur Institute in Paris under the accession number CNCM I-1572,
Lactobacillus paracasei LPC-S01 deposited at Deutsche Sammlung von Mikroorganismen und Zellkulturen GmbH (DSMZ) under accession number DSM 26760,
Bifidobacterium breve BbIBS01 deposited at Deutsche Sammlung von Mikroorganismen und Zellkulturen GmbH (DSMZ) under deposit number DSM 33231,
Bifidobacterium breve BbIBS02 deposited at Deutsche Sammlung von Mikroorganismen und Zellkulturen GmbH (DSMZ) under deposit number DSM 33232,
Bifidobacterium animalis subsp. lactis BIIBS01 and deposited at Deutsche Sammlung von Mikroorganismen und Zellkulturen GmbH (DSMZ) under deposit number DSM 33233,
Lactobacillus plantarum LpIBS01 deposited at Deutsche Sammlung von Mikroorganismen und Zellkulturen GmbH (DSMZ) under deposit number DSM 33234, and
a mixture thereof;
and, optionally, said composition further comprising
at least one acceptable pharmaceutical or food grade additive and/or excipient.

2. The composition according to claim 1, wherein said at least one amino acid is selected from asparagine, alanine, methionine, aspartic acid, histidine, valine, tryptophan, leucine, phenylalanine, isoleucine, tyrosine and mixtures thereof.

3. The composition according to claim 1, wherein said mixture M comprises at least one bacterial strain, or a derivative thereof, selected from the group consisting of: Lactobacillus paracasei DG®CNCM I-1572, Lactobacillus paracasei LPC-S01 DSM 26760, and a mixture thereof.

4. The composition according to claim 3, wherein said mixture M further comprises at least one bacterial strain, or a derivative thereof, selected from:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
a mixture thereof.

5. The composition according to claims 1 to 4, wherein said composition further comprises at least one protein of plant origin, and/or at least one peptide of plant origin, and/or at least one amino acid.

6. The composition according to claim 1, wherein said subject suffers from a disease, symptom and/or disorder related with a protein deficiency or with an increased protein requirement.

7. The composition according to claim 6, wherein said disease, symptom and/or disorder related with said protein deficiency or with said increased protein requirement is selected from: decrease in metabolic efficiency, decrease in corpusculated elements in the blood, changes in healing processes, decrease in muscle strength, decrease in muscle mass, muscle depletion, weight loss, decrease in athletic performance, early fatigue, difficulty in concentrating, anxiety, changes in sleep, changes in mood, increased susceptibility to infections, digestive deficiency, changes in blood sugar, increased cholesterol, changes in blood-chemical parameters, malnutrition syndrome, such as for example Kwashiorkor or biafra, osteoporosis, decrease in at least one protein-based component of an organism selected from nails, hair, skin, enzymes, neurotransmitters, hormones and immunoglobulins.

8. A food product comprising
proteins of plant origin, and
the mixture M comprising at least one bacterial strain selected from the group consisting of:
Lactobacillus paracasei DG® CNCM I-1572,
Lactobacillus paracasei LPC-S01 DSM 26760,
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
a mixture thereof.

9. The food product according to claim 8, wherein said mixture M comprises at least one bacterial strain, or a derivative thereof, selected from the group consisting of:
Lactobacillus paracasei CNCM I-1572,
Lactobacillus paracasei LPC-S01 DSM 26760, and
a mixture thereof;
and, optionally, wherein said mixture M further comprises at least one bacterial strain, or a derivative thereof, selected from the group consisting of:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
a mixture thereof.

10. The food product according to claim 8, wherein said at least one bacterial strain is in a form of coated viable bacterial strain or coated probiotic, paraprobriotic or postbiotic.

11. The food product according to claim 8 for increasing the blood bioavailability of at least one amino acid derived from said proteins of plant origin.

12. A method for increasing blood bioavailability of at least one amino acid derived from a protein of plant origin in a subject taking through oral route said protein of plant origin or said at least one amino acid or a peptide of plant origin comprising said at least one amino acid, the method comprising
administering to the subject the composition of claim 1 in an effective amount to increase bioavailability of the at least one amino acid, said at least one amino acid is selected from: arginine, glutamine, citrulline, serine, asparagine, glycine, threonine, alanine, ornithine, methionine, proline, lysine, aspartic acid, histidine, valine, glutamic acid, tryptophan, leucine, phenylalanine, isoleucine, cysteine, tyrosine and mixtures thereof.

13. A method for increasing the blood bioavailability of at least one amino acid derived from a protein of plant origin in a subject taking through oral route said protein of plant origin or said at least one amino acid or a peptide of plant origin comprising said at least one amino acid, wherein said method comprises
administering the food product of claim 8 in an effective amount to increase bioavailability of the at least one amino acid, said at least one amino acid is selected from: arginine, glutamine, citrulline, serine, asparagine, glycine, threonine, alanine, ornithine, methionine, proline, lysine, aspartic acid, histidine, valine, glutamic acid, tryptophan, leucine, phenylalanine, isoleucine, cysteine, tyrosine and mixtures thereof.

14. A method to treat a subject taking through oral route said protein or at least one amino acid or a peptide of plant origin comprising said at least one amino acid, the method comprising
administering to the subject probiotic bacterial strain Lactobacillus paracasei DG® CNCM I-1572 freeze-dried in powder form, and/or probiotic bacterial strain Lactobacillus paracasei LPC-S01 DSM 26760 freeze-dried in powder form[, in an effective amount for increasing the blood bioavailability of the at least one amino acid derived from a protein of plant or animal origin, in the subject.

15. (canceled)

16. The method of claim 14, wherein the administering further comprises administering at least one bacterial strain, or a derivative thereof, selected from Bifidobacterium breve BbIBS01 DSM 33231, Bifidobacterium breve BbIBS02 DSM 33232, Bifidobacterium animalis subsp. lactis BIIBS01 DSM 33233, Lactobacillus plantarum LpIBS01 DSM 33234, and a mixture thereof.

17. The composition according to claim 2, wherein the Lactobacillus paracasei CNCM I-1572, Lactobacillus paracasei LPC-S01 DSM 26760, and the mixture thereof are comprised at a 1:1 by weight ratio.

18. The composition according to claim 1, wherein said composition further comprises at least one protein of plant origin, and/or at least one peptide of plant origin, and/or at least one amino acid selected from the group consisting of asparagine, alanine, methionine, aspartic acid, histidine, valine, tryptophan, leucine, phenylalanine, isoleucine, tyrosine and mixtures thereof.

19. The food product according to claim 8, wherein said at least one bacterial strain is in a form of coated viable bacterial strain or coated probiotic, paraprobriotic or postbiotic; at least one bacterial strain is in a form selected from the group consisting of: coated viable bacterial strain, tyndallized bacterial strain, sonicated bacterial strain, inactivated bacterial strain, lysate of the bacterial strain, extract of the bacterial strain, a component of the bacterial strain selected from exopolysaccharide, parietal fraction, metabolite and metabolic bioproduct.

20. The method according to claim 12, wherein said subject suffers from a disease, symptom and/or disorder related with a protein deficiency or with an increased protein requirement.

21. The method according to claim 19, wherein said disease, symptom and/or disorder related with said protein deficiency or with said increased protein requirement is selected from: decrease in metabolic efficiency, decrease in corpusculated elements in the blood, changes in healing processes, decrease in muscle strength, decrease in muscle mass, muscle depletion, weight loss, decrease in athletic performance, early fatigue, difficulty in concentrating, anxiety, changes in sleep, changes in mood, increased susceptibility to infections, digestive deficiency, changes in blood sugar, increased cholesterol, changes in blood-chemical parameters, malnutrition syndrome, such as for example Kwashiorkor or biafra, osteoporosis, decrease in at least one protein-based component of an organism selected from nails, hair, skin, enzymes, neurotransmitters, hormones and immunoglobulins.
